(19) United States
(12) Patent Application Publication
(10) Pub. No.: US 2005/0266433 A1
(43) Pub. Date: Dec. 1, 2005

(54) Magnetic device for isolation of cells and biomolecules in a microfluidic environment

(76) Inventors: Ravi Kapur, Boston, MA (US); Mehmet Toner, Wellesley, MA (US); Bruce L. Carvalho, Watertown, MA (US); Tom Barber, Cambridge, MA (US); Lotien R. Huang, Brookline, MA (US)

Correspondence Address: CLARK & ELBING LLP, 101 FEDERAL STREET, BOSTON, MA, 02110, US

(21) Appl. No.: 11/071,679

(22) Filed: Mar. 3, 2005

Related U.S. Application Data
(60) Provisional application No. 60/549,610, filed on Mar. 3, 2004.

(51) Int. Cl. C12Q 1/68; G01N 33/53; C12M 1/34

(52) U.S. Cl. 435/6; 435/287.2; 435/7.1

(57) ABSTRACT

The present invention features a new and useful magnetic device and methods of its use for isolation, enrichment, and purification of cells, proteins, DNA, and other molecules. In general the device includes magnetic regions or obstacles to which magnetic particles can bind. The chemical groups, i.e., capture moieties, on the surface of the magnetic particles may then be used to bind particles, e.g., cells, or molecules of interest from complex samples, and the bound species may then be selectively released for downstream collection or further analysis.

### CROSS-REFERENCE TO RELATED APPLICATIONS

This application claims benefit of U.S. Provisional Application No. 60/549,610, filed Mar. 3, 2004, which is hereby incorporated by reference.

### BACKGROUND OF THE INVENTION

The invention relates to the fields of microfluidics and sorting of particles and molecules.

There are several approaches devised to separate a population of homogeneous cells from complex mixtures, such as blood. These cell separation techniques may be grouped into two broad categories: (1) invasive methods based on the selection of cells fixed and stained using various cell-specific markers; and (2) noninvasive methods for the isolation of living cells using a biophysical parameter specific to a population of cells of interest.

Invasive techniques include fluorescence activated cell sorting (FACS), magnetic activated cell sorting (MACS), and immunomagnetic colloid sorting. FACS is usually a positive selection technique that uses a fluorescently labeled marker to bind to cells expressing a specific cell surface marker. FACS can also be used to permeabilize and stain cells for intracellular markers that can constitute the basis for sorting. It is fast, typically running at a rate of 1,000 to 1,500 Hz, and well established in laboratory medicine. High false positive rates are associated with FACS because of the low number of photons obtained during extremely short dwell times at high speeds. Complicated multiparameter classification approaches can be used to enhance the specificity of FACS, but multianalyte-based FACS may be impractical for routine clinical testing because of the high cost associated with it. The clinical application of FACS is further limited because it requires considerable operator expertise, is laborious, results in cell loss due to multiple manipulations, and the cost of the equipment is prohibitive.

MACS is used as a cell separation technique in which cells that express a specific surface marker are isolated from a mixture of cells using magnetic beads coated with an antibody against the surface marker. MACS has the advantage of being cheaper, easier, and faster to perform as compared with FACS. It suffers from cell loss due to multiple manipulations and handling.

A magnetic colloid system has been used in the isolation of cells from blood. This colloid system uses ferromagnetic nanoparticles that are coated with goat anti-mouse IgG that can be easily attached to cell surface antigen-specific monoclonal antibodies. Cells that are labeled with ferromagnetic nanoparticles align in a magnetic field along ferromagnetic Ni lines deposited by lithographic techniques on an optically transparent surface. This approach also requires multiple cell handling steps including mixing of cells with magnetic beads and separation on the surfaces. It is also not possible to sort out the individual cells from the sample for further analysis.

Noninvasive techniques include charge flow separation, which employs a horizontal crossflow fluid gradient opposing an electric field in order to separate cells based on their characteristic surface charge densities. Although this approach can separate cells purely on biophysical differences, it is not specific enough. There have been attempts to modify the device characteristics (e.g., separator screens and buffer counterflow conditions) to address this major shortcoming of the technique. None of these modifications of device characteristics has provided a practical solution given the expected individual variability in different samples.

Since the prior art methods suffer from high cost, low yield, and lack of specificity, there is a need for a method for depleting a particular type of cell from a mixture that overcomes these limitations.

### SUMMARY OF THE INVENTION

The present invention features a new and useful magnetic device and methods of its use for isolation, enrichment, and purification of cells, proteins, DNA, and other molecules. In general the device includes magnetic regions or obstacles to which magnetic particles can bind. The chemical groups, i.e., capture moieties, on the surface of the magnetic particles may then be used to bind particles, e.g., cells, or molecules of interest from complex samples, and the bound species may then be selectively released for downstream collection or further analysis.

In one aspect, the invention features a device for the separation of one or more desired analytes from a sample. The device includes a first region of magnetic obstacles disposed in a channel, e.g., a microfluidic channel, and a plurality of magnetic particles attached to at least one of the obstacles by a magnetic interaction.

Another device of the invention for the separation of one or more desired analytes from a sample includes a channel having a plurality of magnetic obstacles, wherein the obstacles include a plurality of magnetic particles, e.g., without any underlying support structure, and a capture moiety capable of binding the one or more analytes is attached to the particles. Alternatively, a device for the separation of one or more desired analytes from a sample includes a channel having a plurality of magnetic obstacles, wherein the obstacles include a plurality of magnetic particles, and the magnetic obstacles are disposed such that at least a portion of the one or more analytes cannot pass between the obstacles. In these embodiments, the channel may further include a region of a plurality of magnetic locations, where the magnetic obstacles are attached to the locations by a magnetic interaction.

In any of the above devices, the obstacles are typically ordered in a two-dimensional array, but can also be randomly disposed. The device may further include a second region of magnetic obstacles, e.g., made of a plurality of magnetic particles, or having a plurality of magnetic particles attached by magnetic interaction thereto. The first and second regions can be arranged in series, in parallel, or interspersed. In some embodiments, a capture moiety capable of binding, specifically or not, one or more analytes is attached to the magnetic particles. Exemplary capture moieties include holo-transferrin and an anti-CD71, an anti-CD36, an anti-GPA, or an anti-CD45 antibody, and combinations thereof. When two or more regions of obstacles are employed, different regions may contain different capture moieties to bind two or more different analytes. When capture moieties are employed, the obstacles are typically disposed such that the one or more analytes are capable of passing between the obstacles. When capture moieties are not employed, the obstacles may be disposed such that at least a portion of the one or more analytes cannot pass between the obstacles, e.g., based on size, shape, or deformability.

Other compounds, e.g., cell surface receptors and candidate drug compounds, may also be attached to a magnetic particle, with or without a capture moiety. The attachment of other compounds to magnetic particles allows for the determination of the effect of that compound on an analyte, e.g., effects of candidate drugs on cells, or the identification of ligands for cell surface receptors. The attachment of a plurality of candidate drug compounds or receptors allows for high throughput screening in the device.

In other embodiments, at least a portion of the magnetic obstacles includes a permanent or non-permanent magnet. A device may also include a magnetic force generator capable of producing a magnetic field in the magnetic obstacles, e.g., an electromagnetic or a permanent magnet having a nonuniform magnetic field. Preferably, the magnetic field generator is capable of independently applying the magnetic field to one or more obstacles.

The invention also features a method for retaining a first type of analyte in a sample including providing a sample containing at least a first and a second type of analyte and a device of the invention and introducing the sample into the device, wherein the first type of analyte is retained in the device, e.g., by binding to a capture moiety or being retained based on size, shape, or deformability. Preferably, at least 60% of analytes of the first type in the sample are retained, and at least 70% of analytes of the second type in the sample are not retained. The method may also be altered to retain a third type of analyte in the device as well. Once retained, analytes may be contacted with a labeling moiety. The retained analytes may also be released from the device, e.g., for collection, culturing, or analysis, by interrupting the magnetic interaction holding the magnetic particles in the device, or by disrupting an interaction between the analyte and a capture moiety or the capture moiety and the magnetic particle. When a candidate drug compound is attached to the magnetic particles, the first type of analyte is typically a cell, and the method may further include determining the effect of the candidate drug compound on the cell. Similar methods can be used when cell surface receptors are bound to the magnetic particles as the capture moiety, and putative ligands, agonists, or antagonists are the analytes.

By “analyte” is meant a molecule, other chemical species, e.g., an ion, or particle. Exemplary analytes include cells, viruses, nucleic acids, proteins, carbohydrates, and small organic molecules.

By “capture moiety” is meant a chemical species to which a particle binds. A capture moiety may be a compound coupled to a surface or the material making up the surface. Exemplary capture moieties include antibodies, oligo- or polypeptides, nucleic acids, other proteins, synthetic polymers, and carbohydrates.

By “diluent” is meant any fluid that is miscible with the fluid medium of a sample. Typically diluents are liquids. A diluent, for example, contains agents to alter pH (e.g., acids, bases, or buffering agents) or reagents to chemically modify analytes in a sample (e.g., to label an analyte, conjugate a chemical species to an analyte, or cleave a portion of an analyte) or to effect a biological result (e.g., growth media or chemicals that elicit a cellular response or agents that cause cell lysis). A diluent may also contain agents for use in fixing or stabilizing cells, viruses, or molecules. A diluent may also be chemically or biologically inert.

By “magnetic” is meant possessing hard (permanent) or soft (non-permanent) magnetic properties.

By “microfluidic” is meant having at least one dimension of less than 1 mm. For example, a microfluidic device includes a microfluidic channel having a height, width, or length of less than 1 mm.

By “obstacle” is meant an impediment to flow in a channel, e.g., a protrusion from one surface.

By “particle” is meant an object that does not dissolve in a solution on the time scale of an analysis.

By “type” of analyte is meant a population of analytes, e.g., cells or molecules, having a common property, e.g., the presence of a particular surface antigen. A single analyte may belong to several different types of analytes.

By “specifically binding” a type of analyte is meant binding analytes of that type by a specified mechanism, e.g., antibody-antigen interaction, ligand-receptor interaction, nucleic acid complementarity, protein-protein interaction, charge-charge interaction, and hydrophobic-hydrophobic or hydrophilic-hydrophilic interactions. The strength of the bond is generally enough to prevent detachment by the flow of fluid present when analytes are bound, although individual analytes may occasionally detach under normal operating conditions.

Advantages of the invention include the ability to provide a sorting device that need not be functionalized with environmentally sensitive capture moieties prior to packaging the device, thereby increasing the bandwidth of usable capture moieties; a sorting device that can be functionalized with the capture molecules by the end-user in a simple, rapid and reliable manner enabling customized devices for end-user specific applications; and a sorting device that is more universally functional than the prior art devices.

Other features and advantages will be apparent from the following description and the claims.

### BRIEF DESCRIPTION OF THE DRAWINGS

FIG. 1 is a cross-sectional view of a device of the invention and associated process flow for cell isolation followed by release for off-line analysis according to the present invention.

FIG. 2 is a schematic of the fabrication and functionalization of a device of the invention. The magnetized posts enable post-packaging modification of the device.

FIG. 3 is a schematic of an application of a device of the invention to capture and release CD71+ cells from a complex mixture, such as blood, using monoclonal antibodies to the transferrin (CD71) receptor.

FIG. 4 is a schematic representation of an application of a device of the invention to capture and release CD71+ cells from a complex mixture, such as blood, using holo-transferrin. Holo-transferrin is rich in iron content, commercially available, and has higher affinity constants and specificity of interaction with the CD71 receptor than its counterpart monoclonal antibody.

### DETAILED DESCRIPTION OF THE INVENTION

Device

The invention features a device, typically microfluidic, containing a plurality of magnetic obstacles. In its simplest embodiment, the device includes a channel having magnetic regions to which magnetic particles can magnetically attach to create a textured surface, with which analytes passing through the channel can come into contact. By coating these magnetic particles with appropriate capture moieties it is possible to bind desired analytes through affinity mechanisms. The magnetic particles can serve to texture the channel, and through the appropriate choice of magnetic particle size and shape relative to the dimensions of the channel, it is possible to provide a texture that enhances interactions between the analytes of interest and the magnetic particles. The magnetic particles can be magnetically attached to hard magnetic regions of the channel or to soft magnetic regions that are actuated to produce a magnetic field. In addition, these magnetic particles can be released from defined locations within the channel, e.g., by increasing the overall flow rate of the fluid flowing through the device, decreasing the magnetic field, or through some combination of the two. In one embodiment, a spatially nonuniform permanent magnet or electromagnet may be used to create organized and in some cases periodic arrays of magnetic particles within an otherwise untextured microfluidic channel (Deng et al. Applied Physics Letters, 78, 1775 (2001)). An electromagnetic may be employed to create a non-uniform magnetic field in a device. The non-uniform filed creates regions of higher and lower magnetic field strength, which, in turn, will attract magnetic particles in a periodic arrangement within the device. Other external magnetic fields may be employed to create magnetic regions to which magnetic particles attach. A hard magnetic material may also be used in the fabrication of the device, thereby obviating the need for electromagnets or external magnetic fields. In one embodiment, the device contains a plurality of channels having magnetic regions, e.g., to increase volumetric throughput. Further, these channels may be stacked vertically.

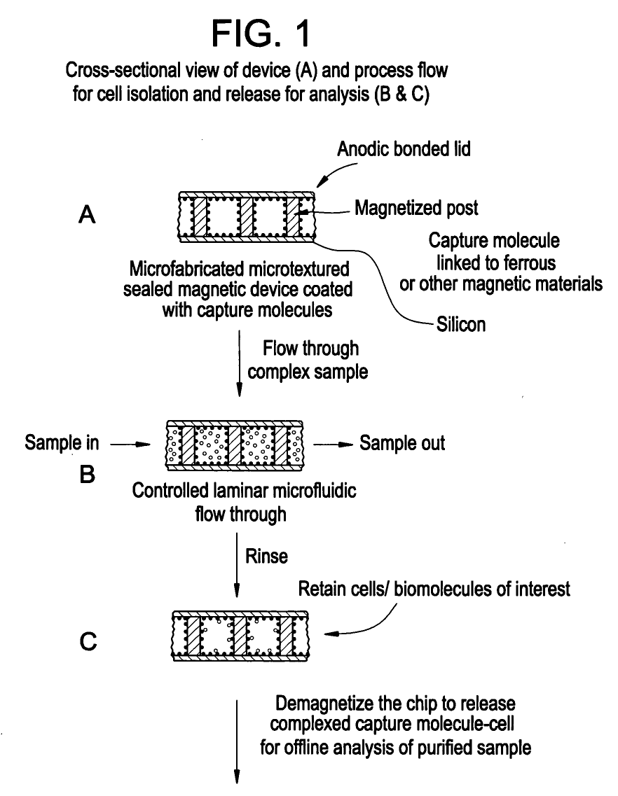

FIG. 1 illustrates an exemplary device geometry and functional process flow to isolate and then release target analytes, e.g., cells or molecules, from a complex mixture. The device contains obstacles that extend from one channel surface toward the opposing channel surface. The obstacles may or may not extend the entire distance across the channel. The obstacles are magnetic (e.g., contain hard or soft magnetic materials or are locations of high magnetic field in a non-uniform field) and attract and retain magnetic particles, which are typically coated with capture moieties. The device geometry, the distribution, shape, size of the posts and the flow parameters can be altered to optimize the efficiency of the interaction of the analytes of interest with the capture moieties (e.g., as described in International Application No. PCT/US03/30965). In one specific example, an anodic lidded silicon wafer with microtextured magnetic obstacles of varying shapes (cylindrical, rectangular, trapezoidal, or pleomorphic) and size (10-999 microns) are arranged uniquely (spacing and density varied across equilateral triangular, diagonal, and random array distribution) to maximize the collision frequency of analytes with the obstacles within the confines of a continuous perfusion flow stream. The exact geometry of the magnetic obstacles and the distribution of obstacles may depend on the type of analytes being isolated, enriched, or purified.

Devices of the invention may or may not include microfluidic channels, i.e., may or may not be microfluidic devices. The dimensions of the channels of the device into which a sample is introduced may depend on the sample employed. Preferably, a channel has at least one dimension (e.g., height, width, length, or radius) of no greater than 10, 9.5, 9, 8.5, 8, 7.5, 7, 6.5, 6, 6.5, 5, 4.5, 4, 3.5, 3, 2.5, 2, 1.5, or 1 mm. Microfluidic devices described herein preferably have channels having at least one dimension of less than 1, 0.9, 0.8, 0.7, 0.6, 0.5, 0.4, 0.3, 0.2, 0.1, or even 0.05 mm. The dimensions of the channels can be determined by one skilled in the art based on the desired application.

Fabrication

A variety of techniques can be employed to fabricate a device of the invention, and the technique employed will be selected based in part on the material of choice. Exemplary materials for fabricating the devices of the invention include glass, silicon, steel, nickel, other metals, poly(methylmethacrylate) (PMMA), polycarbonate, polystyrene, polyethylene, polyolefins, silicones (e.g., poly(dimethylsiloxane)), ceramics, and combinations thereof. Other materials are known in the art. Methods for fabricating channels in these materials are known in the art. These methods include, photolithography (e.g., stereolithography or x-ray photolithography), molding, embossing, silicon micromachining, wet or dry chemical etching, milling, diamond cutting, Lithographie Galvanoformung and Abformung (LIGA), and electroplating. For example, for glass, traditional silicon fabrication techniques of photolithography followed by wet (KOH) or dry etching (reactive ion etching with fluorine or other reactive gas) can be employed. Techniques such as laser micromachining can be adopted for plastic materials with high photon absorption efficiency. This technique is suitable for lower throughput fabrication because of the serial nature of the process. For mass-produced plastic devices, thermoplastic injection molding, and compression molding is suitable. Conventional thermoplastic injection molding used for mass-fabrication of compact discs (which preserves fidelity of features in sub-microns) may also be employed to fabricate the devices of the invention. For example, the device features are replicated on a glass master by conventional photolithography. The glass master is electroformed to yield a tough, thermal shock resistant, thermally conductive, hard mold. This mold serves as the master template for injection molding or compression molding the features into a plastic device. Depending on the plastic material used to fabricate the devices and the requirements on optical quality and throughput of the finished product, compression molding or injection molding may be chosen as the method of manufacture. Compression molding (also called hot embossing or relief imprinting) has the advantages of being compatible with high-molecular weight polymers, which are excellent for small structures, but is difficult to use in replicating high aspect ratio structures and has longer cycle times. Injection molding works well for high-aspect ratio structures but is most suitable for low molecular weight polymers.

A device may be fabricated in one or more pieces that are then assembled. Pieces of a device may be bonded together by clamps, adhesives, heat, anodic bonding, or reactions between surface groups (e.g., wafer bonding). Alternatively, a device may be fabricated as a single piece, e.g., using stereolithography or other three-dimensional fabrication techniques.

Magnetic regions of the device can be fabricated with either hard or soft magnetic materials, such as, but not limited to, rare earth materials, neodymium-iron-boron, ferrous-chromium-cobalt, nickel-ferrous, cobalt-platinum, and strontium ferrite. Portions of the device may be fabricated directly out of magnetic materials, or the magnetic materials may be applied to another material. The use of hard magnetic materials can simplify the design of a device because they are capable of generating a magnetic field without other actuation. Soft magnetic materials, however, enable release and downstream processing of bound analytes simply by demagnetizing the material. Depending on the magnetic material, the application process can include cathodic sputtering, sintering, electrolytic deposition, or thin-film coating of composites of polymer binder-magnetic powder. A preferred embodiment is a thin film coating of micromachined obstacles (e.g., silicon posts) by spin casting with a polymer composite, such as polyimide-strontium ferrite (the polyimide serves as the binder, and the strontium ferrite as the magnetic filler). After coating, the polymer magnetic coating is cured to achieve stable mechanical properties. After curing, the device is briefly exposed to an external induction field, which governs the preferred direction of permanent magnetism in the device. The magnetic flux density and intrinsic coercivity of the magnetic fields from the posts can be controlled by the % volume of the magnetic filler.

In another embodiment, an electrically conductive material is micropatterned on the outer surface of an enclosed microfluidic device. The pattern may consist of a single, electrical circuit with a spatial periodicity of approximately 100 microns. By controlling the layout of this electrical circuit and the magnitude of the electrical current that passes through the circuit, one can develop periodic regions of higher and lower magnetic strength within the enclosed microfluidic device.

The magnetic particles can be disposed uniformly throughout a device or in spatially resolved regions. In addition, magnetic particles may be used to create structure within the device. For example, two magnetic regions on opposite sides of a channel can be used to attract magnetic particles to form a “bridge” linking the two regions.

The magnetic field can be adjusted to influence supra and paramagnetic particles with magnetic mass susceptibility ranging from 0.1-200×10⁻⁶ m³/kg. The paramagnetic particles of use can be classified based on size: particulates (1-5 μm in the size of a cell diameter); colloidal (on the order of 100 nm); and molecular (on the order of 2-10 nm). The fundamental force acting on a paramagnetic entity is:

$${F}_{b} = \frac{1}{2 {μ}_{o}} Δ χ {V}_{G} ∇ {B}^{2}$$

where F_{b} is the magnetic force acting on the paramagnetic entity of volume V_{b}, Δχ is the difference in magnetic susceptibility between the magnetic bead, χb, and the surrounding medium, χf, μₒ is the magnetic permeability of free space, B is the external magnetic field, and ∇ is the gradient operator. The magnetic field can be controlled and regulated to enable attraction and retention of a wide spectrum of particulate, colloidal, and molecular paramagnetic entities typically coupled to capture moieties.
Magnetic Particles and Capture Moieties

Any magnetic particles that respond to a magnetic field may be employed in the devices and methods of the invention. Desirable particles are those that have surface chemistry that can be chemically or physically modified, e.g., by chemical reaction, physical adsorption, entanglement, or electrostatic interaction.

Capture moieties can be bound to magnetic particles by any means known in the art. Examples include chemical reaction, physical adsorption, entanglement, or electrostatic interaction. The capture moiety bound to a magnetic particle will depend on the nature of the analyte targeted. Examples of capture moieties include, without limitation, proteins (such as antibodies, avidin, and cell-surface receptors), charged or uncharged polymers (such as polypeptides, nucleic acids, and synthetic polymers), hydrophobic or hydrophilic polymers, small molecules (such as biotin, receptor ligands, and chelating agents), and ions. Such capture moieties can be used to specifically bind cells (e.g., bacterial, pathogenic, fetal cells, fetal blood cells, cancer cells, and blood cells), organelles (e.g., nuclei), viruses, peptides, protein, polymers, nucleic acids, supramolecular complexes, other biological molecules (e.g., organic or inorganic molecules), small molecules, ions, or combinations or fragments thereof. Specific examples of capture moieties include antiCD71, antiCD36, antiGPA, and holotransferrin. In another embodiment, the capture moiety is fetal cell specific.

Applications

The methods of the invention involve contacting an analyte, for example as a part of a mixture, with the surfaces of a device, and desired analytes (e.g., rare cells such as fetal cells, pathogenic cells, cancer cells, or bacterial cells) in a sample are retained in the device. Analytes of interest may then bind to the surfaces of the device. In another embodiment, desired analytes are retained in the device through size-, shape- or deformability-based separation. Desirably, at least 60%, 70%, 80%, 90%, 95%, 98%, or 99% of the desired analytes are retained in the device. The surfaces of the device are desirably designed to minimize nonspecific binding of non-target analytes. For example, at least 99%, 98%, 95%, 90%, 80%, or 70% of non-target analytes are not retained in the device. The selective retention in the device can result in the separation of a specific analyte population from a mixture, e.g., blood, sputum, urine, and soil, air, or water samples.

The selective retention of desired analytes is obtained by introduction of magnetic particles into a device of the invention. Capture moieties may be bound to the magnetic particles to effect specific binding of the target analyte. Alternatively, the magnetic particles may be disposed such as to only allow analytes of a selected size, shape, or deformability to pass through the device. Combinations of these embodiments are also envisioned. For example, a device may be configured to retain certain analytes based on size and others based on binding. In addition, a device may be designed to bind more than one analyte of interest, e.g., in a serial, parallel, or interspersed arrangement of regions within the device or where two or more capture moieties are disposed on the same magnetic particle or on adjacent particles, e.g., bound to the same obstacle or region. Further, multiple capture moieties that are specific for the same analytes (e.g., antiCD71 and antiCD36) may be employed in the device, either on the same or different magnetic particles, e.g., disposed on the same or different obstacle or region.

Magnetic particles may be attached to obstacles present in the device (or manipulated to create obstacles) to increase surface area for analytes to interact with to increase the likelihood of binding. The flow conditions are typically such that the analytes are very gently handled in the device to prevent damage. Positive pressure or negative pressure pumping or flow from a column of fluid may be employed to transport analytes into and out of the microfluidic devices of the invention. The device enables gentle processing, while maximizing the collision frequency of each analyte with one or more of the magnetic particles. The target analytes interact with any capture moieties on collision with the magnetic particles. The capture moieties can be co-localized with obstacles as a designed consequence of the magnetic field attraction in the device. This interaction leads to capture and retention of the target analytes in defined locations. Alternatively, analytes are retained based on an inability to pass through the device, e.g., based on size, shape, or deformability. Captured analytes can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will. In other embodiments, the particles can be released by disrupting the bond between the analyte and the capture moiety, e.g., through chemical cleavage or interruption of a noncovalent interaction. For example, some ferrous particles are linked to monoclonal antibody via a DNA linker; the use of DNAse can cleave and release the analytes from the ferrous particle. Alternatively, an antibody fragmenting protease (e.g. papain) can be used to engineer selective release. Increasing the sheer forces on the magnetic particles can also be used to release magnetic particles from magnetic regions, especially hard magnetic regions. In other embodiments, the captured analytes are not released and can be analyzed or further manipulated while retained.

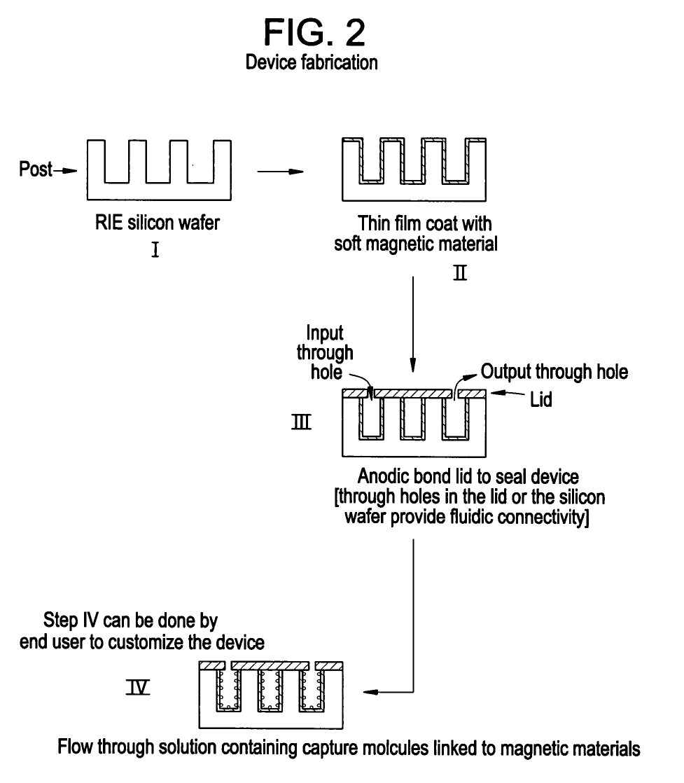

FIG. 2 illustrates the device fabrication and functionalization. The magnetized posts enable post-packaging modification of the device. This is a very significant improvement over existing art. The incompatibility of semiconductor processing parameters (high heat, or solvent sealers to bond the lid) with capture moieties (sensitive to temperature and inorganic and organic solvents) makes this device universal and compatible for functionalization with all capture moieties. Retention of the capture moieties on the obstacles (e.g., posts) by use of magnetic fields, is an added advantage over prior art that uses complex surface chemistry for immobilization. The device enables the end user to easily and rapidly charge the device with a capture moiety, or mixture of capture moieties, of choice thereby increasing the versatility of use. On-demand and ‘just-in-time’ one step functionalization is enabled by this device, thereby circumventing issues of on-the-shelf stability of the capture moieties if they were chemically cross-linked at production. The capture moieties that can be loaded and retained on the posts include, but not limited to, all of the cluster of differentiation (CD) receptors on mammalian cells, synthetic and recombinant ligands for cell receptors, and any other organic, inorganic molecule, or compound of interest that can be attached to any magnetic particle.

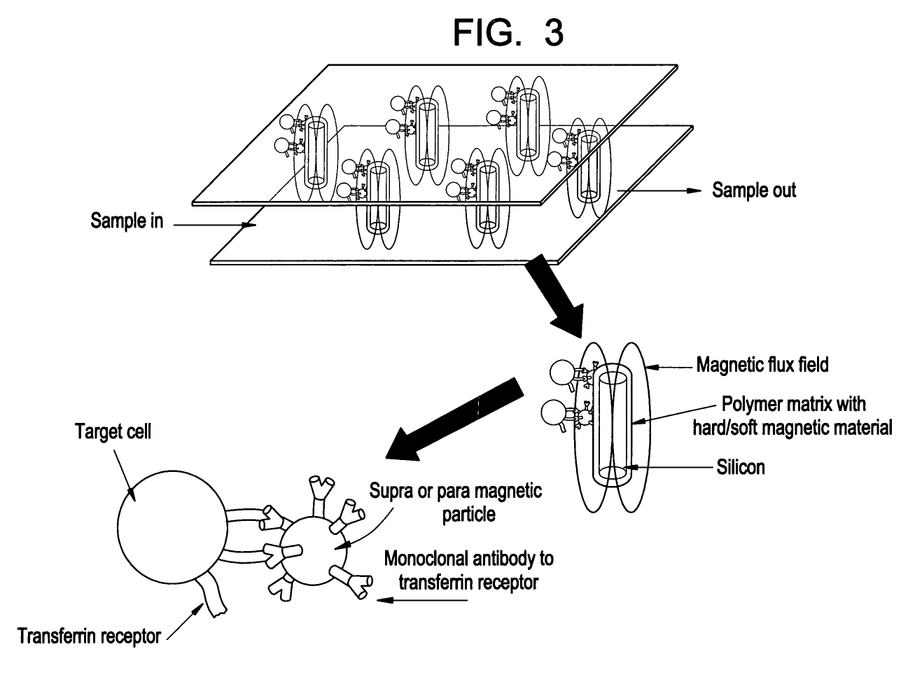

FIG. 3 illustrates an embodiment of the device to capture and isolate cells expressing the transferrin receptor from a complex mixture. Monoclonal antibodies to CD71 receptor are readily available off-the-shelf covalently coupled to magnetic materials, such as, but not limited to ferrous doped polystyrene and ferroparticles or ferro-colloids (e.g., from Miltenyi and Dynal). The mAB to CD71 bound to magnetic particles is flowed into the device. The antibody coated particles are drawn to the posts (i.e., obstacles), floor, and walls and are retained by the strength of the magnetic field interaction between the particles and the magnetic field. The particles between the posts and those loosely retained with the sphere of influence of the local magnetic fields away from the posts, are removed by a rinse (the flow rate can be adjusted such that the hydrodynamic shear stress on the particles away from the posts is larger than the magnetic field strength).

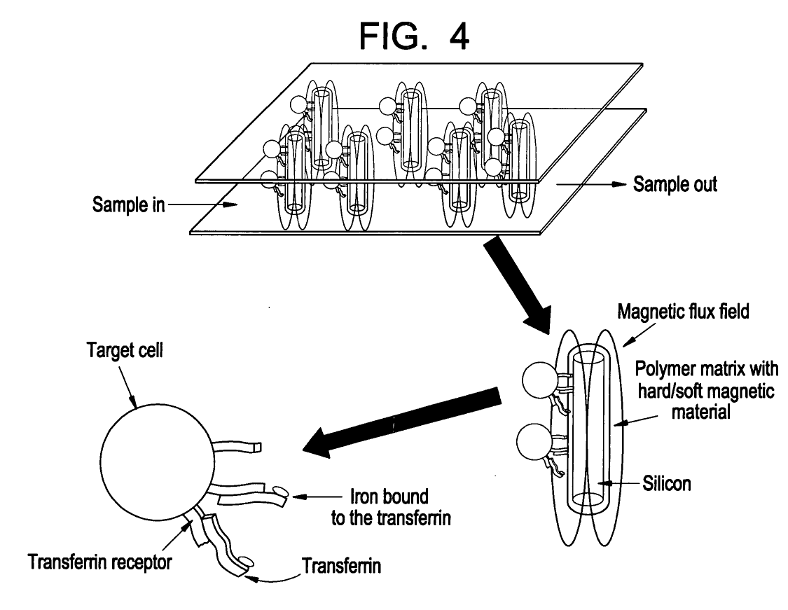

FIG. 4 is a preferred embodiment for application of the device to capture and release CD71+ cells from a complex mixture, e.g., blood, using holo-transferrin. Holo-transferrin is rich in iron content, commercially available, and has higher affinity constants and specificity of interaction with the CD71 receptor than its counterpart monoclonal antibody. The iron coupled to the transferrin ligand serves the dual purpose of retaining the conformation of the ligand for binding with the cell receptor, and as a molecular paramagnetic element for retaining the ligand on the posts.

In addition to the above embodiments, the device can be used for isolation and detection of blood borne pathogens, bacterial and viral loads, airborne pathogens solubilized in aqueous medium, pathogen detection in food industry, and environmental sampling for chemical and biological hazards. Additionally, the magnetic particles can be co-localized with a capture moiety and a candidate drug compound. Capture of a cell of interest can further be analyzed for the interaction of the captured cell with the immobilized drug compound. The device can thus be used to both isolate sub-populations of cells from a complex mixture and assay their reactivity with candidate drug compounds for use in the pharmaceutical drug discovery process for high throughput and secondary cell-based screening of candidate compounds. In other embodiments, receptor-ligand interaction studies for drug discovery can be accomplished in the device by localizing the capture moiety, i.e. the receptor, on a magnetic particle, and flowing in a complex mixture of candidate ligands (or agonists or antagonists). The ligand of interest is captured, and the binding event can be detected, e.g., by secondary staining with a fluorescent probe. This embodiment enables rapid identification of the absence or presence of known ligands from complex mixtures extracted from tissues or cell digests or identification of candidate drug compounds.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A device for retaining one or more desired analytes in a sample, said device comprising a first region of magnetic obstacles disposed in a channel and a plurality of magnetic particles attached to at least one of said obstacles by a magnetic interaction.

2. The device of claim 1, wherein said channel is a microfluidic channel.

3. The device of claim 1, wherein said magnetic particles comprise a capture moiety capable of binding said one or more analytes.

4. The device of claim 3, wherein said capture moiety specifically binds a first type of analyte.

5. The device of claim 4, wherein said capture moiety comprises holo-transferrin or an anti-CD71, an anti-CD36, an anti-GPA, or an anti-CD45 antibody, or a combination thereof.

6. The device of claim 1, further comprising a second region of magnetic obstacles, wherein a plurality of magnetic particles is attached by magnetic interaction to at least one of said obstacles in said second region.

7. The device of claim 6, wherein said obstacles in said first region are interspersed among said obstacles in said second region.

8. The device of claim 6, wherein said obstacles in said first region specifically bind a first type of analyte and said obstacles in said second region specifically bind a second type of analyte.

9. The device of claim 1, wherein at least a portion of said magnetic obstacles comprise a permanent magnet.

10. The device of claim 1, wherein at least a portion of said magnetic obstacles comprise a non-permanent magnet.

11. The device of claim 1, further comprising a magnetic force generator capable of producing a magnetic field in said magnetic obstacles.

12. The device of claim 11, wherein the magnetic field generator is capable of independently applying the magnetic field to one or more obstacles.

13. The device of claim 1, wherein said obstacles are ordered in a two-dimensional array.

14. The device of claim 1, wherein said obstacles are disposed such that said one or more analytes are capable of passing between said obstacles.

15. The device of claim 1, wherein said obstacles are disposed such that at least a portion of said one or more analytes cannot pass between said obstacles.

16. The device of claim 1, wherein said one or more analytes comprise a cell.

17. The device of claim 1, wherein said one or more analytes comprise a molecule.

18. A device for retaining one or more desired analytes in a sample, said device comprising a channel comprising a plurality of magnetic obstacles, wherein said obstacles comprise a plurality of magnetic particles, and said magnetic particles comprise a capture moiety capable of binding said one or more analytes.

19. A device for retaining one or more desired analytes in a sample, said device comprising a channel comprising a plurality of magnetic obstacles, wherein said obstacles comprise a plurality of magnetic particles, and wherein said magnetic obstacles are disposed such that at least a portion of said one or more analytes cannot pass between said obstacles.

20. The device of claim 18, wherein said channel further comprises a region of a plurality of magnetic locations and said magnetic obstacles are attached to said locations by a magnetic interaction

21. The device of claim 18, wherein said one or more analytes comprise a cell.

22. A method for retaining a first type of analyte in a sample, said method comprising the steps of:
(a) providing a sample comprising at least a first and a second type of analyte and a device comprising:
(i) a first region of magnetic obstacles disposed in a channel; and
(ii) a plurality of magnetic particles attached to at least one of said obstacles by a magnetic interaction; and
(b) introducing said sample into said device, wherein said first type of analyte is retained in said device by interaction with at least one of said obstacles.

23. A method for retaining a first type of analyte in a sample, said method comprising the steps of:
(a) providing a sample comprising at least a first and a second type of analyte and a device comprising a first region of magnetic obstacles disposed in a channel, wherein said obstacles comprise a plurality of magnetic particles; and
(b) introducing said sample into said device, wherein said first type of analyte is retained in said device by interaction with at least one of said obstacles.

24. The method of claim 22, wherein said magnetic particles are coated with a capture moiety capable of binding said first type of analyte.

25. The method of claim 22, wherein said magnetic obstacles are disposed such that at least a portion of said first type of analyte cannot pass between said obstacles.

26. The method of claim 22, wherein said first type of analyte is a particle

27. The method of claim 26, wherein said particle is a cell.

28. The method of claim 27, wherein said cell is bacterial cell, a fetal cell, or a blood cell.

29. The method of claim 22, wherein said particle is an organelle.

30. The method of claim 29, wherein said organelle is a nucleus.

31. The method of claim 22, wherein said particle is a virus.

32. The method of claim 22, wherein said first type of analyte is a molecule.

33. The method of claim 32, wherein said molecule is a nucleic acid, protein, or supramolecular complex.

34. The method of claim 22, wherein at least 60% of analytes of said first type in said sample are retained.

35. The method of claim 22, wherein at least 70% of analytes of said second type in said sample are not retained.

36. The method of claim 22, wherein said capture moiety comprises holo-transferrin or an anti-CD71, an anti-CD36, an anti-GPA, or an anti-CD45 antibody, or a combination thereof.

37. The method of claim 24, wherein said capture moiety comprises an antibody, a protein, a peptide, or a nucleic acid.

38. The method of claim 22, wherein said device further comprises a second region of magnetic obstacles having magnetic particles attached by magnetic interaction thereto, and wherein said magnetic particles attached to said obstacles in said second region are coated with a capture moiety that selectively binds a third type of analyte.

39. The method of claim 38, wherein said obstacles in said first region are interspersed among said obstacles in said second region.

40. The method of claim 22, wherein said magnetic obstacles comprise a permanent magnet.

41. The method of claim 22, wherein said magnetic obstacles comprise a non-permanent magnet.

42. The method of claim 22, wherein said device further comprises a magnetic force generator capable of producing a magnetic field in said magnetic obstacles.

43. The method of claim 42, wherein the magnetic field generator is capable of applying the magnetic field to one or more obstacles independently.

44. The method of claim 22, wherein said obstacles are ordered in a two-dimensional array.

45. The method of claim 22, further comprising contacting a labeling moiety with said first type of analyte retained in said device

46. The method of claim 22, further comprising interrupting the magnetic interaction thereby releasing said first type of analyte from said obstacles.

47. The method of claim 22, wherein said channel is a microfluidic channel.

48. The method of claim 24, wherein said first type of analyte is specifically bound to said capture moiety.

49. The method of claim 24, wherein a candidate drug compound is attached to said magnetic particles.

50. The method of claim 49, wherein said first type of analyte is a cell, and further comprising determining the effect of said candidate drug compound on said first type of cell bound to said capture moiety.

51. The method of claim 24, wherein said capture moiety comprises a cell surface receptor.

52. The method of claim 51, wherein said sample comprises candidate ligands for said cell surface receptor.

53. The method of claim 24, wherein said device further comprises a second plurality of magnetic particles attached to at least one of said obstacles by a magnetic interaction, wherein said magnetic particles are coated with a second capture moiety capable of binding said first type of analyte.

54. The method of claim 53, wherein said plurality of particles in step (a)(ii) and said second plurality of particles are disposed on the same obstacle.
